(19) United States
(12) Patent Application Publication
(10) Pub. No.: US 2022/0372479 A1
(43) Pub. Date: Nov. 24, 2022

(54) Oligonucleotide based ex vivo cell therapy

(71) Applicant: Secarna Pharmaceuticals GmbH & Co. KG, Marburg (DE)

(72) Inventors: Frank JASCHINSKI, Marburg (DE); Richard KLAR, Marburg (DE)

(21) Appl. No.: 17/765,170

(22) PCT Filed: Oct. 5, 2020
(86) PCT No.: PCT/EP2020/077778

§ 371 Date: Mar. 30, 2022

(30) Foreign Application Priority Data
Oct. 4, 2019 (EP) 19201432.2

(51) Int. Cl. C12N 15/113; C12N 5/078; C12N 5/074; A61K 35/15

(52) U.S. Cl. CPC C12N 15/113; C12N 5/0634; C12N 5/0696; A61K 35/15

(57) ABSTRACT

The present invention refers to a method for reducing expression of a target RNA in an isolated cell in preparation for cell therapy, comprising incubating the isolated cell comprising the target RNA with an antisense oligonucleotide without use of a transfection means, wherein the antisense oligonucleotide is administered to the isolated cell at least once in a time period of day 0 to day 21, the antisense oligonucleotide hybridizes with the target RNA and reduces the transcription of the target RNA, reduces the expression of the protein encoded by the target RNA or a combination thereof up to 8 weeks from day 0 of the incubation with the antisense oligonucleotide. The invention further relates to an isolated cell obtainable by the method of the present invention and a pharmaceutical composition comprising the isolated cell. The isolated cell and the pharmaceutical composition are used in a method of preventing and/or treating a disease.

The present invention refers to an ex vivo method for reducing a target RNA in an isolated cell in preparation for use in cell therapy, to the isolated cell obtainable via this method and to a pharmaceutical composition comprising the isolated cell. The isolated cell and the pharmaceutical composition are for use in a method of preventing and/or treating a disease.

### TECHNICAL BACKGROUND

Cell therapy (also called cellular therapy or cytotherapy) is therapy in which cellular material is injected, grafted or implanted into a patient, i.e., it is injected, grafted or implanted in intact, living cells. The cells may originate from the patient (autologous cells) or a donor (allogeneic cells). The cells used in cell therapy can be classified by their potential to transform into different cell types. Pluripotent cells can transform into any cell type in the body and multipotent cells can transform into other cell types, but their repertoire is more limited than that of pluripotent cells. Differentiated or primary cells are of a fixed type. The type of cells administered depends on the treatment. For example, T cells capable of fighting cancer cells via cell-mediated immunity may be injected in the course of immunotherapy. Cell therapy is targeted at many clinical indications in multiple organs and by several modes of cell delivery. Accordingly, the specific mechanisms of action involved in the therapies are wide-ranging.

Stem cell or progenitor cell engraftment, differentiation, and long term replacement of damaged tissue for example. In this paradigm multipotent or unipotent cells differentiate into a specific cell type in the lab or after reaching the site of injury (via local or systemic administration). These cells then integrate into the site of injury, replacing damaged tissue, and thus facilitate improved function of the organ or tissue. An example of this is the use of cells to replace cardiomyocytes after myocardial infarction.

Cells that have the capacity to release soluble factors such as cytokines, chemokines, and growth factors which act in a paracrine or endocrine manner. These factors facilitate self-healing of the organ or region. The delivered cells (via local or systemic administration) remain viable for a relatively short period (days-weeks) and then die. This includes cells that naturally secrete the relevant therapeutic factors, or which undergo epigenetic changes or genetic engineering that causes the cells to release large quantities of a specific molecule. Examples of this include cells that secrete factors which facilitate angiogenesis, anti-inflammation, and anti-apoptosis.

Cells such as immune cells, blood cells or skin cells can usually reproduce ex vivo given the right conditions. This allows differentiated, adult immune cells to be used for cell therapy. The cells can be removed from the body, isolated from a mixed cell population, modified and then expanded before return to the body. A recently developed cell therapy involves the transfer of adult self-renewing T lymphocytes which are genetically modified to increase their immune potency to kill disease-causing cells.

Potential applications of cell therapies include treating cancers, autoimmune disease, urinary problems, and infectious disease, rebuilding damaged cartilage in joints, repairing spinal cord injuries, improving a weakened immune system, or helping patients with neurological disorders.

The downsides are unsatisfying activities and thus, unsatisfying results of the different cell therapies and that cell therapy often causes severe side effects—some of them life-threatening—which must be managed by experienced specialists. These side effects occur for example when modified cells rapidly proliferate and release a flood of substances called cytokines. Severe cytokine release syndrome can lead to life-threatening multi-organ damage and brain swelling. Thus, modified cells used in cell therapy have the capacity to elicit expected and unexpected toxicities including cytokine release syndrome, neurologic toxicity, “on target/off tumor” recognition, and anaphylaxis.

Thus, there is an urgent need to develop cell therapy having reduced side effects and at least the efficiency of existing cell therapies or even increased efficiency.

This present invention satisfies this need.

### SUMMARY

The present invention refers to a method for reducing expression of a target RNA in an isolated cell in preparation for cell therapy, comprising:

incubating the isolated cell comprising the target RNA with an antisense oligonucleotide without use of a transfection means, wherein the antisense oligonucleotide is administered to the isolated cell at least once in a time period of day 0 to day 21, the antisense oligonucleotide hybridizes with the target RNA and reduces the transcription of the target RNA, reduces the transcription of the target RNA, reduces the expression of the protein encoded by the target RNA or a combination thereof up to 8 weeks from day 0 of the incubation with the antisense oligonucleotide. The target RNA is for example selected from the group consisting of mRNA, pre-mRNA, lncRNA, and/or miRNA.

The cell of the present invention is for example an immune cell. The immune cell is for example selected from the group consisting of a T cell, a dendritic cell, a natural killer (NK) cell, peripheral blood mononuclear cell (PBMC), a stem cell such as a hematopoietic stem cell and/or an induced pluripotent stem cell, a B cell and a combination thereof. Alternatively or additionally, the immune cell is a T-cell and the target RNA is for example encoding a protein that affects efficacy and/or safety of the immune cell selected from the group consisting of PD-1, TIGIT, TIM-3, LAG-3, TGFBR, CD39, CD73, 2B4, BTLA, VISTA, CD304, PQR-prot, Chop, XBP1, PERK, FOXP3, GMCSF, IFNg, TNFa, TGFb, IL-1, IL-2, IL-6, IL-10, IL-12, IL-17, IL-9, STAT3, IL-6 receptor and a combination thereof, or the target RNA is for example encoding a protein that affects expansion and/or survival of the immune cell selected from the group consisting of BID, BIM, BAD, NOXA, PUMA, BAX, BAK, BOK, BCL-rambo, BCL-Xs, Hrk, Blk, BMf, p53 and a combination thereof.

The isolated cell is for example genetically modified by a gene transfer technology before or after incubating the cell with the antisense oligonucleotide. Optionally, the isolated, genetically modified cell such as an immune cell is expanded before or after incubating the cell with the antisense oligonucleotide.

The method for reducing expression of a target RNA in the isolated cell in preparation for use in cell therapy of the present invention further optionally comprises the step of purifying the isolated cell before and/or after incubating the cell such as an immune cell with the antisense oligonucleotide.

Optionally, the method, further comprises the step of concentrating the isolated cell such as an immune cell before and/or after incubating the immune cell with the antisense oligonucleotide, wherein optionally antisense oligonucleotide is added to the isolated immune cell.

Optionally, the method further comprises the step of cryopreserving the isolated cell such as an immune cell when incubated with the antisense oligonucleotide, before incubating the immune cell with the antisense oligonucleotide, after incubating the cell with the antisense oligonucleotide or a combination thereof.

In case of an isolated immune cell used in the method for reducing expression of a target RNA in the isolated cell in preparation for use in cell therapy the cell is selected from the group consisting of a T-cell, or a dendritic cell, a natural killer (NK) cell, peripheral blood mononuclear cell (PBMC), a stem cell such as a hematopoietic stem cell and/or an induced pluripotent stem cell, a B cell and a combination thereof.

The target RNA is for example encoding a protein that affects efficacy and/or safety of the immune cell such as a dendritic cell, a natural killer (NK) cell, peripheral blood mononuclear cell (PBMC), a stem cell such as a hematopoietic stem cell and/or an induced pluripotent stem cell, a B cell and a combination thereof selected from the group consisting of PD-1, TIGIT, TIM-3, LAG-3, TGFBR, CD39, CD73, 2B4, BTLA, VISTA, CD304, PQR-prot, Chop, XBP1, PERK, FOXP3, GMCSF, IFNg, TNFa, TGFb, IL-1, IL-2, IL-6, IL-10, IL-12, IL-17, IL-9, STAT3, IL-6 receptor and a combination thereof, or the target RNA is for example encoding a protein that affects expansion and/or survival of the immune cell selected from the group consisting of BID, BIM, BAD, NOXA, PUMA, BAX, BAK, BOK, BCL-rambo, BCL-Xs, Hrk, Blk, BMf, p53 and a combination thereof.

In the method of the present invention, the antisense oligonucleotide is for example administered in a time period of day 0 to day 21, of day 0 to day 20, of day 0 to day 19, of day 0 to day 18, of day 0 to day 17, of day 0 to day 16, of day 0 to day 15, of day 0 to day 14, of day 0 to day 13, of day 0 to day 12, of day 0 to day 11, of day 0 to day 10, of day 0 to day 9, of day 0 to day 8, of day 0 to day 7, of day 0 to day 6, of day 0 to day 5, of day 0 to day 4, of day 0 to day 3, of day 0 to day 2 or of day 0 to day 1. The antisense oligonucleotide is for example administered every day, every second day, every third day, every fourth day, every fifth day, every sixth day, every seventh day, every eighth day, every ninth day or every tenth day of the time period.

Optionally, in the method of the present invention the antisense oligonucleotides hybridize for example with two or more target RNAs which are for example administered at the same time points for the same time periods or at different time points for the same or different time periods.

The present invention further relates to a cell such as an isolated immune cell for use in a method of preventing and/or treating a disease, wherein the isolated immune cell originates from a patient suffering from the disease or a healthy subject, and is incubated ex vivo with an antisense oligonucleotide hybridizing with a target RNA according to the method of the present invention to reduce expression of the target RNA, and after incubation of the isolated immune cell with the antisense oligonucleotide, the isolated immune cell is reintroduced into the patient or introduced into a patient. Hence, the immune cell is obtainable by a method of the present invention and used in a method of preventing and/or treating a disease.

In addition, the present invention is directed to a pharmaceutical composition comprising an isolated immune cell of the present invention for use in a method of preventing and/or treating a disease and a pharmaceutically acceptable excipient. A patient suffering from the disease and/or a healthy subject is for example a human or non-human animal. The disease is for example selected from the group consisting of cancer, autoimmune disease, graft-versus-host disease and a combination thereof.

All documents cited or referenced herein (“herein cited documents”), and all documents cited or referenced in herein cited documents, together with any manufacturer's instructions, descriptions, product specifications, and product sheets for any products mentioned herein or in any document incorporated by reference herein, are hereby incorporated herein by reference, and may be employed in the practice of the invention. More specifically, all referenced documents are incorporated by reference to the same extent as if each individual document was specifically and individually indicated to be incorporated by reference.

### DESCRIPTION OF THE FIGURES

FIG. 1 depicts potent knockdown of target RNA independent of initial seeding density of isolated cells.

FIG. 2 shows reduction of target RNA expression after cryopreservation based on previous antisense oligonucleotide incubation.

FIG. 3 depicts parallel reduction of expression of different target RNAs via parallel incubation with two different antisense oligonucleotides in dendritic cells.

FIG. 4A depicts parallel reduction of expression of different target RNAs and FIG. 4B shows parallel reduction of expression of different proteins via parallel incubation with two different antisense oligonucleotides in T cells.

FIG. 5 shows superior target RNA reduction of a target-specific antisense oligonucleotide in T cells as compared to target-specific siRNA.

### DETAILED DESCRIPTION

The present invention relates to a method for reducing expression of a target RNA in an isolated cell such as an immune cell in preparation for use in cell therapy. The method comprises the steps of incubating the isolated cell such as an immune cell comprising the target RNA with an antisense oligonucleotide without use of a transfection means such as gymnotic transfection. The antisense oligonucleotide is administered to the isolated cell such as an immune cell at least once in a time period of day 0 to day 21. The antisense oligonucleotide hybridizes with the target RNA and reduces the transcription of the target RNA, reduces the expression of the protein encoded by the target RNA or a combination thereof up to 8 weeks from day 0 of the incubation with the antisense oligonucleotide. As the administration of the antisense oligonucleotides of the present invention does not permanently block the transcription, function and/or expression of the target RNA side effects are avoided which are based on permanent blocking of RNA transcription, function and/or expression. Additionally, administration of an antisense oligonucleotide without transfection means significantly reduces the stress on a cell and reduces or even avoids side effects caused by other transfection means.

In the following, the elements of the present invention will be described in more detail. These elements are listed with specific embodiments, however, it should be understood that they may be combined in any manner and in any number to create additional embodiments. The variously described examples and embodiments should not be construed to limit the present invention to only the explicitly described embodiments. This description should be understood to support and encompass embodiments which combine the explicitly described embodiments with any number of the disclosed elements. Furthermore, any permutations and combinations of all described elements in this application should be considered disclosed by the description of the present application unless the context indicates otherwise.

Throughout this specification and the claims, unless the context requires otherwise, the word “comprise”, and variations such as “comprises” and “comprising”, will be understood to imply the inclusion of a stated member, integer or step or group of members, integers or steps but not the exclusion of any other member, integer or step or group of members, integers or steps. The terms “a” and “an” and “the” and similar reference used in the context of describing the invention (especially in the context of the claims) are to be construed to cover both the singular and the plural, unless otherwise indicated herein or clearly contradicted by the context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”, “for example”), provided herein is intended merely to better illustrate the invention and does not pose a limitation on the scope of the invention otherwise claimed. No language in the specification should be construed as indicating any non-claimed element essential to the practice of the invention.

A cell of the present invention is for example an immune cell, a stem cell, a pluripotent stem cell such as an induced pluripotent stem cell, an embryonic stem cell, a skin stem cell, a cord blood stem cell, a mesenchymal stem cell, a neural stem cell or a combination thereof. The immune cell is for example selected from the group consisting of a T-cell, or a dendritic cell, a natural killer (NK) cell, a peripheral blood mononuclear cell (PBMC), a stem cell such as a hematopoietic stem cell and/or an induced pluripotent stem cell, a B cell and a combination thereof.

If a T-cell is selected, the T-cell expresses one or more factors for example selected from the group consisting of PD-1, TIGIT, TIM-3, LAG-3, TGFBR, CD39, CD73, 2B4, BTLA, VISTA, CD304, PQR-prot, Chop, XBP1, PERK, FOXP3, GMCSF, IFNg, TNFa, TGFb, IL-1, IL-2, IL-6, IL-10, IL-12, IL-17, IL-9, STAT3, IL-6 receptor, BID, BIM, BAD, NOXA, PUMA, BAX, BAK, BOK, BCL-rambo, BCL-Xs, Hrk, Blk, BMf, p53 and any combination thereof. If a T-cell is selected, the target RNA encodes for example a protein that affects efficacy and/or safety of the T-cell selected from the group consisting of PD-1, TIGIT, TIM-3, LAG-3, TGFBR, CD39, CD73, 2B4, BTLA, VISTA, CD304, PQR-prot, Chop, XBP1, PERK, FOXP3, GMCSF, IFNg, TNFa, TGFb, IL-1, IL-2, IL-6, IL-10, IL-12, IL-17, IL-9, STAT3, IL-6 receptor and a combination thereof, or encodes a protein that affects expansion and/or survival of the T-cell selected from the group consisting of BID, BIM, BAD, NOXA, PUMA, BAX, BAK, BOK, BCL-rambo, BCL-Xs, Hrk, Blk, BMf, p53 and a combination thereof.

Immune cells such as T cells, dendritic cells, natural killer cells, peripheral blood mononuclear cells, stem cells such as a hematopoietic stem cells and/or an induced pluripotent stem cell, B cells are for example genetically modified to express an antigen-specific receptor such as a chimeric antigen receptor or an immune cell receptor such as a T cell receptor. Those cells can exert their anti-tumor function by recognizing an antigen on the surface of a tumor cell via the antigen-specific receptor, which leads to activation of the immune cell such as a T cell. The activated immune cell such as a T cell for example releases cytokines and toxic molecules that lead to destruction of the tumor cell.

Reducing expression of a target RNA according to the present invention means decreasing the transcription, function and/or expression of a target RNA in different amounts up to complete inhibition. The transcription, function and/or expression level in the cell is determined for example by measuring and comparing the transcription, function and/or expression level of the target RNA before treatment, i.e., administration of an oligonucleotide, and after treatment.

The target RNA is for example mRNA, pre-mRNA, lncRNA, and/or miRNA. The oligonucleotide hybridizes with a specific sequence of the target RNA and reduces the transcription, function and/or expression of the target RNA consisting of or comprising this sequence. The target RNA is for example directly or indirectly involved in the causing and/or maintenance of a disease. The target RNA affects for example directly or indirectly an increase in proliferation, a decrease in exhaustion, faster cell growth, an increase in metabolic activity, an improvement in functionality, an improvement of the immunomodulatory effect, and increase in efficiency, and/or increase in potency of a secretome. The method of the present invention results in the reduction of the amount of the target RNA in the cell such as an immune cell.

lncRNA for example can function as signal, decoy, scaffold, guide, enhancer RNAs, and even as short peptide. The main function of a signal lncRNA is for example to serve as a molecular signal to regulate transcription in response to various stimuli. Decoy lncRNAs for example limit the availability of regulatory factors by presenting “decoy” binding sites. The lncRNA for example modulates transcription by sequestering regulatory factors including transcription factors, catalytic proteins, subunits of larger chromatin modifying complexes, as well as miRNAs, thereby reducing their availability. Transcripts from the scaffold class of lncRNAs play for example a structural role by providing platforms for assembly of multiple-component complexes, such as ribonucleoprotein (RNP) complexes. Guide lncRNAs interact with RNPs and direct them for example to specific target genes. These guide lncRNAs are for example essential for the proper localization of RNPs. Enhancer RNAs (eRNAs) are produced from enhancer regions and influence for example the 3-dimensional (3D) organization of DNA, known as “chromatin interactions”. In addition, lncRNAs encode for example short regulatory peptides.

The target RNA encodes for example a protein that affects efficacy and/or safety of the immune cell selected from the group consisting of PD-1, TIGIT, TIM-3, LAG-3, TGFBR, CD39, CD73, 2B4, BTLA, VISTA, CD304, PQR-prot, Chop, XBP1, PERK, FOXP3, GMCSF, IFNg, TNFa, TGFb, IL-1, IL-2, IL-6, IL-10, IL-12, IL-17, IL-9, STAT3, IL-6 receptor and a combination thereof, or encodes a protein that affects expansion and/or survival of the immune cell selected from the group consisting of BID, BIM, BAD, NOXA, PUMA, BAX, BAK, BOK, BCL-rambo, BCL-Xs, Hrk, Blk, BMf, p53 and a combination thereof.

The oligonucleotide has a direct and/or an indirect effect: It hybridizes with the target RNA expressing a factor of interest, e.g., PD-1, TIGIT, TIM-3, LAG-3, TGFBR, CD39, CD73, 2B4, BTLA, VISTA, CD304, PQR-prot, Chop, XBP1, PERK, FOXP3, GMCSF, IFNg, TNFa, TGFb, IL-1, IL-2, IL-6, IL-10, IL-12, IL-17, IL-9, STAT3, IL-6 receptor, BID, BIM, BAD, NOXA, PUMA, BAX, BAK, BOK, BCL-rambo, BCL-Xs, Hrk, Blk, BMf, p53 or a combination thereof (direct effect), and/or it hybridizes with the target RNA of another factor which is influencing, e.g., inhibiting or activating the factor of interest (indirect effect).

The oligonucleotide of the present invention is for example an antisense oligonucleotide, siRNA or aptamer. The oligonucleotide comprises for example one or more modifications such as a bridged nucleotide, e.g., a locked nucleic acid (LNA, e.g., 2′,4′-LNA), cET, ENA, a 2′Fluoro modified nucleotide, a 2′O-Methyl modified nucleotide, a morpholino or a combination thereof.

Reduction of the target RNA in the isolated cell is for example causative for higher proliferation, less exhaustion, faster growth, more potent secretome, higher metabolic activity, better functionality, improved immunomodulatory effect, less side effects, favorable cytokine profile and/or a higher efficacy of and in the isolated cell, respectively.

The cell used in the method of the present invention is for example isolated from a human or non-human animal. The human animal is for example a human being of any genetic background; non-human animal comprises mammalian such as horse, cattle, pig, lamb, cat, dog, guinea pig, hamster etc.; fish such as trout, salmon, zander; bird such as goose, duck, ostrich etc. of any genetic background.

The isolated cell is optionally genetically modified by a gene transfer technology including 1) transfection by (bio)chemical methods, 2) transfection by physical methods and 3) virus-mediated transduction. (Bio)chemical methods are for example calcium phosphate transfection, transfection with DEAE-dextran, or lipofection; physical methods are for example electroporation, nucleofection, microinjection, transfection by particle bombardment or transfection by ultrasound; and virus-mediated transduction uses for example adenoviruses for short-term infections with high-level transient expression, herpesviruses for long-term expression or retroviruses for stable integration of DNA into the host cell genome. Following the genetic modification the cell is expanded.

The isolated cell is for example incubated with the oligonucleotide before or after the genetic modification and/or before or after the expansion of the genetically modified cell. Optionally, the isolated cell is purified, e.g., by one or more washing steps, before and/or after incubation with the oligonucleotide.

The method of the present invention optionally comprises a concentrating step, wherein the isolated cell is concentrated via any concentration method of the art before and/or after the incubation with the oligonucleotide. An oligonucleotide is for example administered to the isolated cell again after the concentrating step.

Further, the isolated cell is for example cryopreserved when incubated with the oligonucleotide, before incubation with the oligonucleotide and/or after incubation with the oligonucleotide, after any purification step, after any concentrating step or a combination thereof. The cryopreserved isolated cell such as an immune cell surprisingly maintains the antisense oligonucleotide-mediated knockdown efficacy of the target RNA. The efficacy of cryopreserved versus non-cryopreserved isolated cells in decreasing or increasing transcription and/or translation of target RNA and/or in reduction of target RNA is highly comparable.

Isolation according to the present invention means obtaining cells from a source, e.g., immune cells from blood, stem cell from bone marrow or blood of the umbilical cord etc., and/or obtaining a subpopulation of cells from a previously isolated cells or a cell population.

Purification according to the present invention means cleansing of an isolated cell from an undesired substance such as an extracellular substance for example via High Pressure Liquid Chromatography (HPLC), Fast Protein Liquid Chromatography (FPLC) etc.

The method of the present invention optionally comprises an activation step, wherein the isolated cell is activated via any activation method of the art for example by stimulating the cell using monoclonal antibodies specific for CD3 or CD23 on the surface of T cells, or by stimulating a B cell using CD40 ligand (CD40L) before and/or after the incubation with the oligonucleotide. An oligonucleotide is for example administered to the isolated cell again after the activation step.

The method of the present invention optionally comprises an expansion step, wherein the isolated cells is expanded via any expansion method of the art for example by adding basic fibroblast growth factor (FGF2) to mesenchymal stem cells before and/or after the incubation with the oligonucleotide or by adding interleukin-2 (IL-2) and/or interleukin-(IL-15) to NK cells before and/or after the incubation with the oligonucleotide.

The method of the present invention optionally comprises a differentiation step, wherein the isolated cell is for example a monocyte that is differentiated into an immature DC by adding interleukin-4 (IL-4) and granulocyte macrophage colony stimulating factor (GM-CSF) to the cell before and/or after the incubation with the oligonucleotide.

The method of the present invention optionally comprises a maturation step, wherein the isolated cells is for example an immature DC, which is for example a natural immature DC or an artificially immature DC, e.g., originating from a differentiation step of a monocyte as described above, that is matured into a mature DC by adding a toll-like receptor ligand such as R848 or LPS or by adding a cytokine such as interferon gamma (IFN-γ).

The isolated cell is incubated with the oligonucleotide for a time period (incubation period) of for example day 0 to day 21, of day 0 to day 20, of day 0 to day 19, of day 0 to day 18, of day 0 to day 17, of day 0 to day 16, of day 0 to day 15, of day 0 to day 14, of day 0 to day 13, of day 0 to day 12, of day 0 to day 11, of day 0 to day 10, of day 0 to day 9, of day 0 to day 8, of day 0 to day 7, of day 0 to day 6, of day 0 to day 5, of day 0 to day 4, of day 0 to day 3, of day 0 to day 2 or of day 0 to day 1. Day 0 is the day when the first oligonucleotide is added the first time to the isolated cell. The oligonucleotide is for example added only once to the isolated cell, or every day during the time period or every second day, every third day, every fourth day, every fifth day, every sixth day, every seventh day, every eighth day, every ninth day, every tenth day of the time period or only on the first and the last day of the time period, which represent administration patterns. During the incubation period any administration pattern can be combined, e.g., the incubation period is day 0 to day 9, where the oligonucleotide is administered for five days every day and for four days every second day. After the time period the oligonucleotide is for example removed from the isolated cell. The oligonucleotide is added to the isolated cell in a nanomolar or micromolar range for example 0.1 nmol to 1000 μmol, 0.5 nmol to 900 μmol, 1 nmol to 800 μmol, 50 nmol to 700 μmol, 100 nmol to 600 μmol, 200 nmol to 500 μmol, 300 nmol to 400 μmol, 500 nmol to 300 μmol, 600 nmol to 200 μmol, 700 nmol to 100 μmol, or 800 nmol to 50 μmol.

The oligonucleotide reduces the expression of the target RNA for example for at least 10 weeks, for at least 8 weeks, for at least 6 weeks or for at least 4 weeks from day 0 of the incubation period. The reduction of the expression of the target RNA is for example independent of the incubation period with the oligonucleotide. These reduction terms of the expression of the target RNA are reached with each of the above mentioned incubation periods.

The isolated cell is for example incubated with one or more, e.g., 2, 3, 4, 5, 6, 7, 8, 9 or 10 different oligonucleotides. The different oligonucleotides are administered to the isolated cell at the same time point for the same time period, at the same time point for different time periods, at different time points for the same period or at different time points for different time periods.

Alternatively or in addition, the target RNA is one or more target RNAs, i.e., the same oligonucleotide for example reduces the expression of more than one target RNA, different oligonucleotides reduce the expression of different target RNAs, e.g., in parallel or subsequently having a direct and/or indirect effect on the factor of interest.

The present invention is further directed to an isolated cell such as an isolated immune cell obtainable by the method of the present invention. The isolated, e.g., immune cell is for example for use in a method of preventing and/or treating a disease. The cell is for example isolated from a patient suffering from the disease or from a healthy subject and the isolated cell is incubated ex vivo with an oligonucleotide hybridizing with a target RNA according to the method of the present invention. After incubating the isolated cell such as an isolated immune cell with the oligonucleotide, the isolated cell is reintroduced into the patient from whom it was isolated. Alternatively, the cell isolated from a healthy subject and incubated ex vivo with an oligonucleotide hybridizing with a target RNA according to the method of the present invention is introduced into a patient. The patient suffering from a disease is treatable by a cell treated with an oligonucleotide such as an antisense oligonucleotide that targets an RNA that affects for example directly or indirectly an increase in proliferation, a decrease in exhaustion, faster cell growth, an increase in metabolic activity, an improvement in functionality, an improvement of the immunomodulatory effect, and increase in efficiency, and/or increase in potency of a secretome. Thus, the present invention comprises allogenic cell therapy. The oligonucleotide for example is reintroduced or introduced into the patient intravenously, intraperitoneally, intramuscularly and/or subcutaneously.

The cell such as an immune cell for use in a method of preventing and/or treating a disease comprises isolated cells such as isolated immune cells from a patient, a healthy subject or a combination thereof, which have been incubated ex vivo with an antisense oligonucleotide hybridizing with a target RNA according to the present invention.

The invention further relates to a pharmaceutical composition comprising the isolated cell of the invention and a pharmaceutically acceptable excipient. The pharmaceutical composition is for example for use in a method of preventing and/or treating a disease, wherein the pharmaceutical composition is for example administered intravenously, intraperitoneally, intramuscularly or subcutaneously. The administration of the isolated cell or the pharmaceutical composition is for example based on an infusion or an injection.

The disease is for example cancer, autoimmune disease, graft-versus-host disease, stroke, spinal cord injury, bone disease, age-related macular degeneration, Parkinson's disease, amyotrophic lateral sclerosis, Alzheimer disease, diabetes and a combination thereof.

### EXAMPLES

The following examples illustrate different embodiments of the present invention, but the invention is not limited to these examples. In the following experiments no transfecting agent is used, i.e., gymnotic delivery is performed.

### Example 1: Knockdown Efficacy in Human T Cells without the Use of a Transfection Reagent is Independent of Cell Seeding Density

CD3+ human T cells were seeded in 96-well u-bottom plates at different densities, namely 20.000, 50.000, 75.000 and 100.000 per well, on day 0 and treated with 5 μM of a target-specific antisense oligonucleotide or a control oligonucleotide on day 0 and day 3.

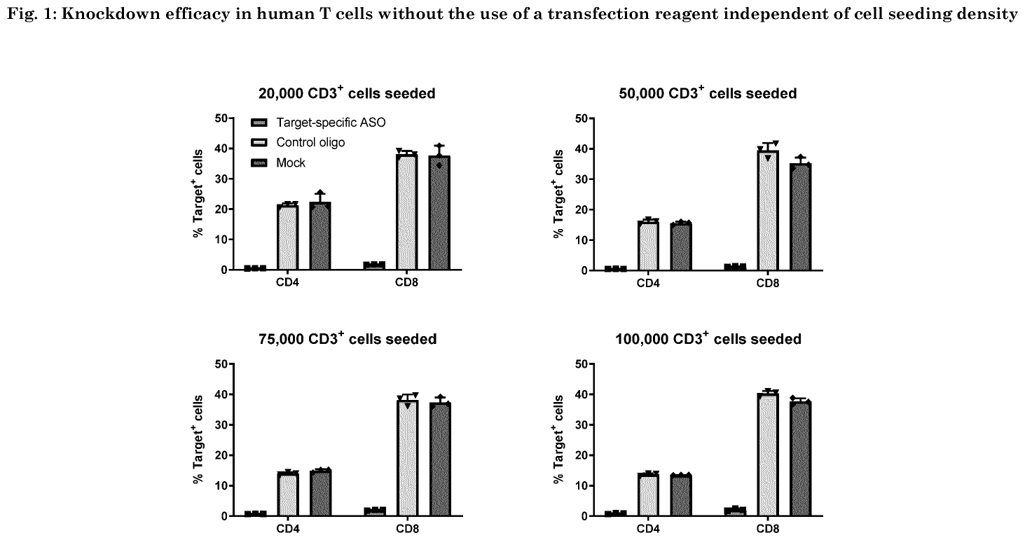

Target protein expression was investigated on day 6 by flow cytometry. Potent target knockdown could be achieved independently of the initial seeding density. The results are shown in FIG. 1.

### Example 2: Knockdown Persists Over Several Days after Removal of the Antisense Oligonucleotide and Cryopreservation of the Cells

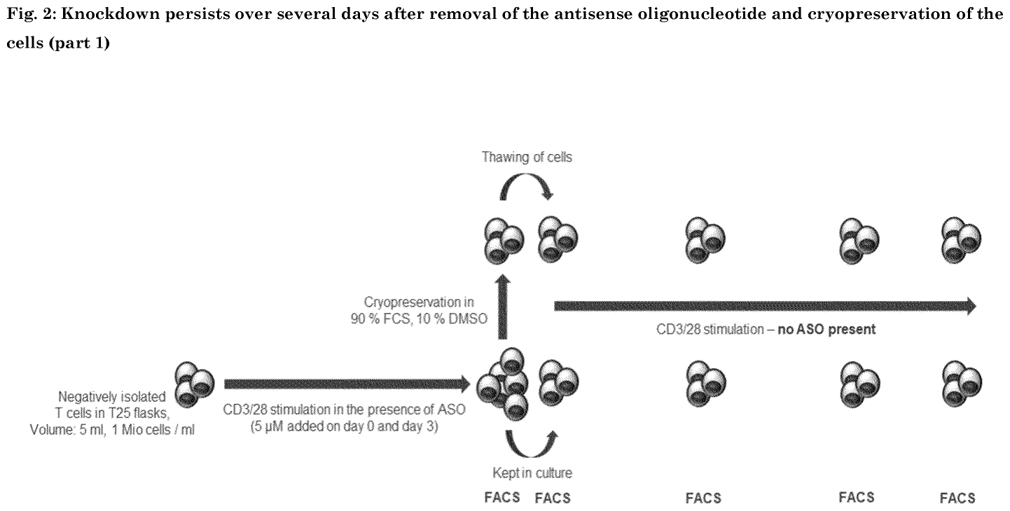

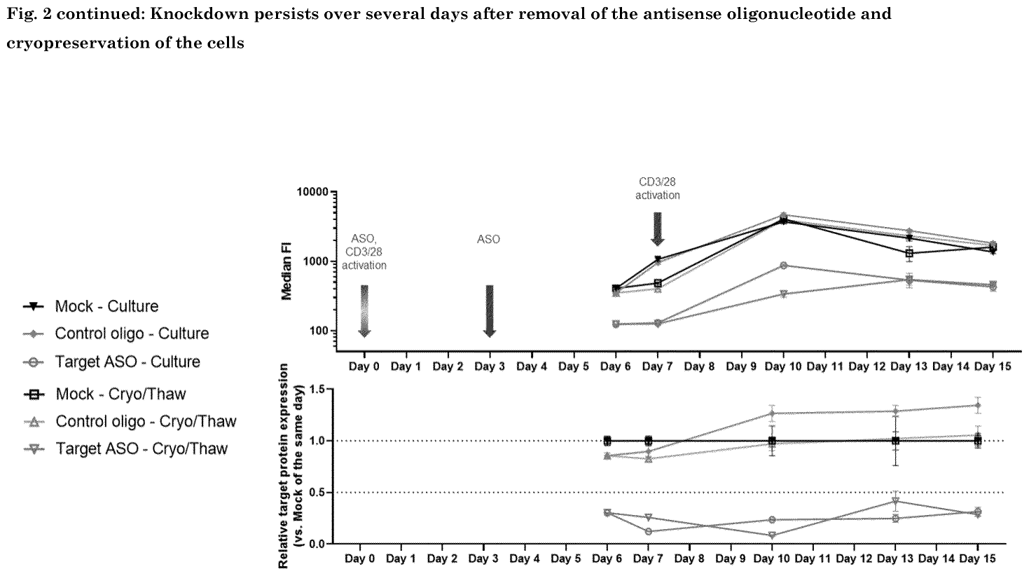

CD3+ human T cells were seeded in T25 flasks, activated and treated with mock, a control oligo or a target-specific antisense oligonucleotide for six days. Part of the cells was then kept in culture (“Culture”), the other part was cryopreserved on day six. Cryopreserved cells were thawed (“Cryo/Thaw”) on day seven and conditions “Culture” and “Cryo/Thaw” were restimulated without addition of control oligo or target-specific antisense oligonucleotide. Target expression was measured over time on the protein level until eight days after restimulation. Potent target knockdown could be measured at all tested time points and there was no difference between conditions “Culture” and “Cryo/Thaw”. Therefore, antisense oligonucleotide-mediated knockdown persists throughout a cryopreservation/thawing process as shown in FIG. 2.

### Example 3: Simultaneous Knockdown of Two Targets in Dendritic Cells

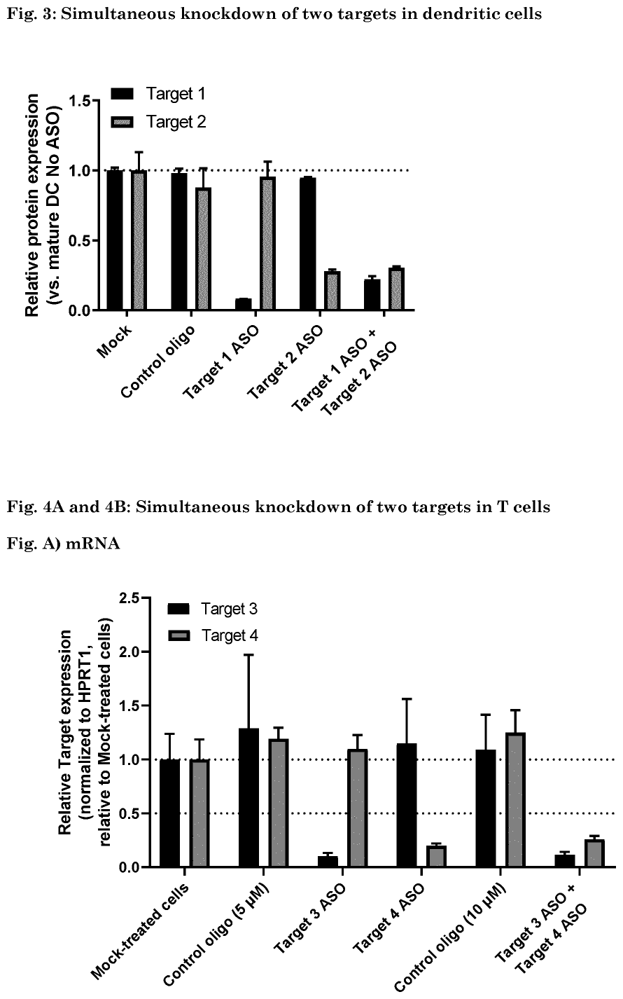

CD14+ monocytes were differentiated into mature dendritic cells (DC) and treated with either a control oligo, a target 1-specific antisense oligonucleotide, a target 2-specific antisense oligonucleotide or the combination of target 1- and target 2-specific antisense oligonucleotide. Target protein expression was analyzed on day 3. Potent target knockdown of target 1 and 2 could be observed in the respective monotherapy settings. Strikingly, potent knockdown of both targets could be achieved when cells were treated with a combination of target 1- and target 2-specific antisense oligonucleotide. Results are depicted in FIG. 3.

### Example 4: Simultaneous Knockdown of Two Targets in T Cells

T cells were isolated and activated in order to induce the expression of Target 3 and Target 4 on day 0. A control oligonucleotide (neg1, final conc.: 5 μM), a Target 3-specific antisense oligonucleotide (final conc.: 5 μM), a Target 4-specific antisense oligonucleotide (final conc.: 5 μM), a combination of Target 3- and Target 4-specific antisense oligonucleotide (final conc.: 5 μM each) or a control oligonucleotide (neg1, final conc.: 10 μM) was added to the cells on day 0. Target mRNA and HPRT1 mRNA (housekeeper) expression was analyzed on day 3 by the Quantigene SinglePlex assay (ThermoFisher) according to the manufacturer's instructions. Target expression values were normalized for HPRT1 expression values and relative expression compared to mock-treated cells was calculated.

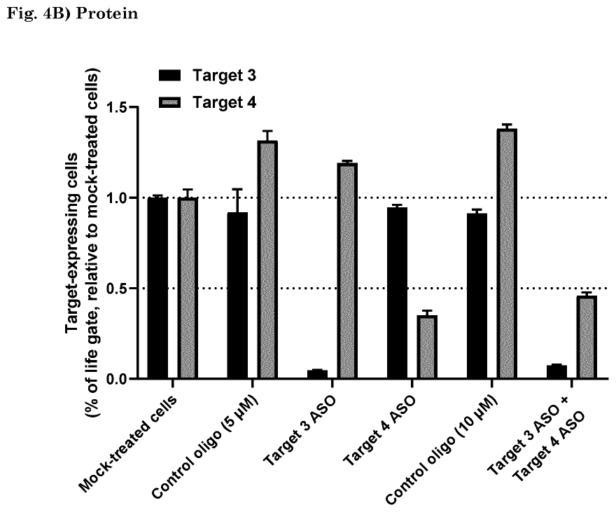

The percentage of Target protein-expressing cells was investigated on day 5 by flow cytometry. The relative percentage of Target protein-expressing cells as compared to mock-treated cells is depicted. A potent knockdown of Target 3 using a Target 3-specific antisense oligonucleotide and a potent knockdown of Target 4 using a Target 4-specific antisense oligonucleotide was observed. Strikingly, when Target 3 and Target 4 antisense oligonucleotides were added to the cells in combination, knockdown of both targets was observed with an efficacy that was comparable to the conditions where cells had been treated with the respective antisense oligonucleotide alone. Results are shown in FIG. 4A (mRNA) and FIG. 4B (protein).

### Example 5: Comparison of a Target 3-Specific siRNA with a Target 3-Specific Antisense Oligonucleotide

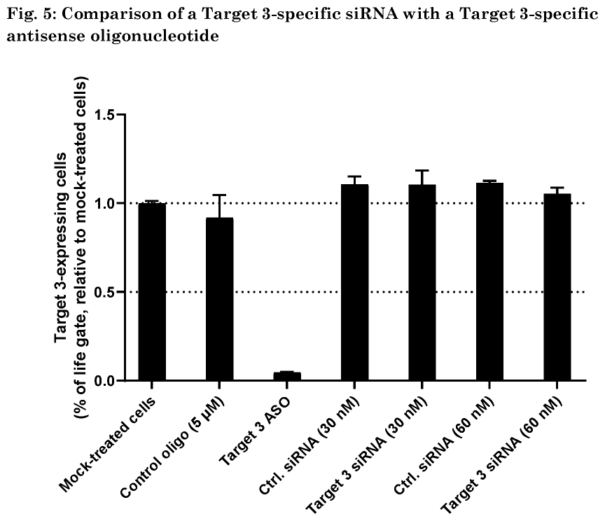

T cells were isolated and activated in order to induce the expression of Target 3 on day 0. A control oligonucleotide (neg1, final conc. 5 μM) or a Target 3-specific antisense oligonucleotide (final conc. 5 μM) were added to the cells on day 0 without the use of a transfection reagent. As comparison, either a non-targeting siRNA (Ambion, final conc.: 30 nM and 60 nM) or a Target 3-specific siRNA (Ambion, final conc.: 30 nM and 60 nM) were transfected into T cells on day 0 using a standard transfection protocol with Lipofectamine 2000 (ThermoFisher). The percentage of Target 3 protein-expressing cells was investigated on day 5 by flow cytometry. The relative percentage of Target 3 protein expressing cells as compared to mock-treated cells is depicted. A potent Target 3 knockdown was observed, when cells had been treated with a Target 3-specific antisense oligonucleotide. In contrast, there was no reduction of Target 3-expressing cells, when cells had been transfected with a Target 3-specific siRNA at both tested concentrations (30 nM and 60 nM). Results are shown in FIG. 5.

1. Method for reducing expression of a target RNA in an isolated immune cell selected from the group consisting of a dendritic cell, a natural killer (NK) cell, a peripheral blood mononuclear cell (PBMC), a stem cell such as a hematopoietic stem cell and/or an induced pluripotent stem cell, a B cell and a combination thereof in preparation for use in cell therapy, comprising:
incubating the isolated immune cell comprising the target RNA with an antisense oligonucleotide without use of a transfection means, wherein the antisense oligonucleotide is administered to the isolated immune cell at least once in a time period of day 0 to day 21, the antisense oligonucleotide hybridizes with the target RNA and reduces the transcription of the target RNA, reduces the function of the target RNA, reduces the expression of the protein encoded by the target RNA or a combination thereof up to 8 weeks from day 0 of the incubation with the antisense oligonucleotide.

2. Method according to claim 1, wherein the target RNA is selected from the group consisting of mRNA, pre-mRNA, lncRNA, and/or miRNA.

3. Method of claim 1 or 2, wherein the isolated immune cell is genetically modified by a gene transfer technology before or after incubating the immune cell with the antisense oligonucleotide.

4. Method of claim 3, wherein the isolated, genetically modified immune cell is expanded before or after incubating the immune cell with the antisense oligonucleotide.

5. Method according to any one of claims 1 to 4, further comprising the step of purifying the isolated immune cell before and/or after incubating the immune cell with the antisense oligonucleotide.

6. Method according to any one of claims 1 to 5, further comprising the step of concentrating the isolated immune cell before and/or after incubating the immune cell with the antisense oligonucleotide, wherein optionally antisense oligonucleotide is added to the isolated immune cell again after the concentrating step.

7. Method according to any one of claims 1 to 6, further comprising the step of cryopreserving the isolated immune cell when incubated with the antisense oligonucleotide, before incubating the immune cell with the antisense oligonucleotide, after incubating the immune cell with the antisense oligonucleotide or a combination thereof.

8. Method according to any one of claims 1 to 7, wherein the target RNA is encoding a protein that affects efficacy and/or safety of the immune cell selected from the group consisting of PD-1, TIGIT, TIM-3, LAG-3, TGFBR, CD39, CD73, 2B4, BTLA, VISTA, CD304, PQR-prot, Chop, XBP1, PERK, FOXP3, GMCSF, IFNg, TNFa, TGFb, IL-1, IL-2, IL-6, IL-10, IL-12, IL-17, IL-9, STAT3, IL-6 receptor and a combination thereof, or wherein the target RNA is encoding a protein that affects expansion and/or survival of the immune cell selected from the group consisting of BID, BIM, BAD, NOXA, PUMA, BAX, BAK, BOK, BCL-rambo, BCL-Xs, Hrk, Blk, BMf, p53 and a combination thereof.

9. Method according to any one of claims 1 to 8, wherein the antisense oligonucleotide is administered in a time period of day 0 to day 20, of day 0 to day 19, of day 0 to day 18, of day 0 to day 17, of day 0 to day 16, of day 0 to day 15, of day 0 to day 14, of day 0 to day 13, of day 0 to day 12, of day 0 to day 11, of day 0 to day 10, of day 0 to day 9, of day 0 to day 8, of day 0 to day 7, of day 0 to day 6, of day 0 to day 5, of day 0 to day 4, of day 0 to day 3, of day 0 to day 2 or of day 0 to day 1.

10. Method according to any one of claims 1 to 9, wherein the antisense oligonucleotide is administered every day, every second day, every third day, every fourth day, every fifth day, every sixth day, every seventh day, every eighth day, every ninth day or every tenth day of the time period.

11. Method according to any one of claims 1 to 10, wherein antisense oligonucleotides hybridizing with two or more target RNAs are administered at the same time points for the same or different time periods or at different time points for the same or different time periods.

12. Isolated immune cell for use in a method of preventing and/or treating a disease, wherein the isolated immune cell originates from a patient suffering from the disease or a healthy subject and is incubated ex vivo incubation with an antisense oligonucleotide hybridizing with a target RNA according to the method of any one of claims 1 to 11 to reduce expression of the target RNA, and after incubation of the isolated immune cell with the antisense oligonucleotide, the isolated immune cell is reintroduced into the patient or introduced into a patient.

13. Pharmaceutical composition comprising a cell for use according to claim 12 and a pharmaceutically acceptable excipient.

14. Immune cell for use according to claim 12 or pharmaceutical composition according to claim 13, wherein the patient and/or the healthy subject is a human or non-human animal.

15. Immune cell for use according to claim 12 or 14, or pharmaceutical composition according to claim 13 or 14, wherein the disease is selected from the group consisting of cancer, autoimmune disease, graft-versus-host disease and a combination thereof.
